(19) United States
(12) Patent Application Publication
(10) Pub. No.: US 2008/0050499 A1
(43) Pub. Date: Feb. 28, 2008

(54) Process for preparing a composition comprising steroidal glycosides

(75) Inventors: Frederik Michiel Meeuse, Vlaardingen (NL); Kevin John Povey, Purfleet (GB); Christopher Swindells, Cambs (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/893,088

(22) Filed: Aug. 14, 2007

(30) Foreign Application Priority Data
Aug. 24, 2006 (EP) EP06119457

(51) Int. Cl. A23L 1/39; A01H 5/00; A61K 31/704; A61P 3/04; A23L 1/00

(52) U.S. Cl. 426/589; 426/640; 426/655; 514/26; 800/298

(57) ABSTRACT

Process for preparing a composition comprising one or more steroidal glycosides, comprising the steps of:
(a) harvesting Hoodia plants,
(b) cutting up the harvested plants,
(c) drying the cut plants,
   whereby exposure to UV light during the drying step is avoided, such that the total UV dose is less than about 0.5 kJ/m² to obtain dried plant material.

### TECHNICAL FIELD

The present invention relates generally to the field of steroidal glycosides. More in particular, it relates to a process for preparing a composition comprising steroidal glycosides from the plants of the Hoodia genus (formerly the Hoodia and Trichocaulon genera), which can be used for example in weight management products.

### BACKGROUND OF THE INVENTION

Extracts obtainable from plants of the Asclepiadaceae family, particularly the Hoodia genus (formerly the Hoodia and Trichocaulon genera) have been shown to have an appetite suppressant activity and are potentially useful in weight management products. U.S. Pat. No. 6,376,657 (CSIR) discloses that these extracts contain steroidal glycosides having the formula 1:

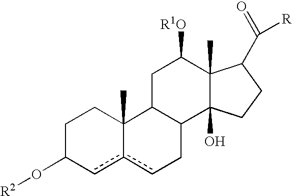

wherein

### R=alkyl;

### R¹═H, alkyl, tiglyol, benzoyl or any other organic ester group;

R²═H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

US '657 also discloses a process to extract the steroidal glycoside having the formula 1 from plants of the Asclepiadaceae family, involving treating plant material with a solvent to extract a fraction having appetite suppressant activity, separating the extraction solution from the rest of the plant material, removing the solvent from the extraction solution and recovering the extract. The patent also discloses methods for synthesizing various steroidal glycosides.

WO2005/116049 (Unilever) discloses that steroidal glycosides can be extracted or separated from undesirable components present in plant material of the Asclepiadaceae (Hoodia) family by means of liquid or supercritical carbon dioxide. Dried plant material from Hoodia gordonii was milled to a fine powder and subsequently extracted.

US 2005/0202103 discloses Caralluma extracts, wherein the aerial parts of Caralluma plant are dried under shade (on cemented platform).

U.S. Pat. No. 7,008,648 discloses a method of obtaining a plant material from Stapelia and Orbea plants, wherein a suitable method for drying and grinding the original biomass includes either sun drying followed by a heated air-drying or freeze-drying, e.g. lyophilization or chopping of the biomass into small pieces, e.g. 2-10 cm, followed by heated air-drying or freeze-drying.

There is still a need for alternative or improved processes for preparing a composition comprising one or more steroidal glycosides, especially for increasing the content of steroidal glycosides in the dried plant. Furthermore, the yield of some specific steroidal glycosides may be adversely affected by the process conditions.

It is therefore an object of the invention to provide a process for preparing a composition comprising one or more steroidal glycosides, which does not have one or more of the above mentioned drawbacks.

It was now surprisingly found that that the drying conditions, in particular the exposure to UV light, have an important influence on the yield of steroidal glycosides.

### Definition Of The Invention

The above and other objects are attained by the present invention, which includes, in its first aspect,

A process of preparing a composition comprising one or more steroidal glycosides, comprising the steps of:

(a) harvesting Hoodia plants,
(b) cutting up the harvested plants,
(c) drying the cut plants, whereby exposure to UV light during the drying step is avoided, such that the total UV dose is less than 0.5 kJ/m2 to obtain dried plant material.

In a second aspect, the invention relates to dried plant material obtainable according to the above process and having a mean total steroidal glycosides content of at least 1.3% by weight, or an individual plant total steroidal glycoside content of at least 1.6%. The dried plant material obtainable according to the invention comprises the steroidal glycoside of Formula 2—described hereinbelow—in an amount of at least 0.08% by weight (calculated as a mean), or an individual level of steroidal glycoside of Formula 2 of at least 0.08% by weight.

In a third aspect, the invention relates to extracts and food products comprising the steroidal glycosides.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.”

It should be noted that in specifying any range of concentration or amount, any particular upper concentration can be associated with any particular lower concentration or amount.

For the avoidance of doubt the word “comprising” is intended to mean “including” but not necessarily “consisting of” or “composed of.” In other words, the listed steps or options need not be exhaustive.

“Cut” as used herein means that the size of the plant is reduced, and includes comminuting, pulverising, etc.

“Steroidal glycoside” as used herein means a steroid (four fused rings), further comprising at least one side group substitution which is a glycoside (a molecule in which a sugar group is bonded through its anomeric carbon to another group via an O-glycosidic bond), preferably a deoxy or di-deoxy glycoside and includes all steroidal glycosides eluting after 15 minutes as described in HPLC Steroidal Glycoside Analysis hereinbelow.

Steroidal glycoside amounts herein are based on anhydrous plant material (the moisture level in the dried plant is measured and the steroidal glycoside amount is normalised to 0% moisture).

“Mean” as used herein means the average steroidal glycoside content of at least 10 different, randomly selected, plants.

“Total UV dose” as used herein means the Erythemal UV level integrated over the drying period. The Erythemal UV is defined by the CIE (Commission Internationale de l'Éclairage, International Commission on Illumination) action spectrum (E), using the method described by McKinlay, A. F. and B. L. Diffey (“A reference action spectrum for ultraviolet induced erythema in human skin, in Human Exposure to Ultraviolet Radiation: Risks and Regulations”, pp. 83-87, Elsevier, Amsterdam, 1987):

### E(λ)=1 when 280 nm<λ<298 nm,

### E(λ)=10^{0.094(298-λ)} when 298 nm <λ<328 nm,

### E(λ)=10^{0.015(139-λ)} when 328 nm <λ<400 nm.

The daily erythemal UV dose is the erythemal UV level integrated over the entire day. Websites are available that show daily this daily erythemal UV dose for any location in the world (www.temis.nl from the European Space Agency).

Alternatively one can use a UV sensor which measures the UV radiation in the range from 280 to 400 nm. The radiation levels then need to be integrated with the erythemal action spectrum as defined above. Then this erythemal UV level needs to be integrated over the drying period to obtain the UV dose.

The first aspect of the present invention is a process for preparing a composition comprising one or more steroidal glycosides, in particular those obtainable from plants of the Hoodia family. More preferably, said plant is selected from the group consisting of Trichocaulon piliferum, Trichocaulon officinale, Hoodia currorii, Hoodia gordonii, Hoodia lugardii and mixtures thereof. Hoodia gordonii is especially preferred.

In the first step of the process, the plants are harvested.

Subsequently, the harvested plants are cut. The plant can be cut into any shape, like cubes, slices, julliene, etc as long as one of the dimensions is less then 30 mm, preferably less then 20 mm, most preferably less then 15 mm. So for a slice or julliene shape the thickness should be less then these dimensions, for a cube all dimensions should be less then these dimensions, etc. Coventional cutting equipment may be used such as a wood chipper, a bowl cutter or standard food cutting equipment, for example the machines supplied by Urschell to form plant particles. The smaller the size, the faster the drying time, reducing the potential for microbial growth. The whole plants may be used, but preferably the plants are used without roots, to minimize the potential for microbial contamination.

The cut plants are then dried under conditions whereby exposure to UV light is minimized. This is counterintuitive, since in the regions where the plants such as Hoodia prosper, the sun is the cheapest form of energy for drying processes. In fact, Rooibos tea is traditionally made in South Africa by drying the plants in the sun on a cement floor.

The UV dose varies with the geographical locations and season of the year. For instance in South Africa the maximum daily UV dose is about 7 kJ/m2 (summer time), whereas the minimum UV dose is about 2 kJ/m2 (winter time). Shade structures can be constructed to shield the UV radiation. The UV protection of a shade structure can be quantified with the UV protection factor (UPF). A UPF of 15 means that 1/15th of the incoming UV is transmitted, the rest is reflected. A typical shade cloth has a UPF between 2 and 10. The dose under shade depends heavily on the type of shade structure. Moreover surfaces like water, snow, sand and concrete reflect UV radiation such that the actual UV levels increase. For instance, concrete surface increases the UV level. Moreover when the drying takes two days, the total UV dose the material is exposed to is double that level.

In the present invention, it was found that the direct exposure to UV light should not exceed 0.5 kJ/m2, preferably not exceed 0.35, more preferably not exceed 0.2 kJ/m2, most preferably not exceed 0.1 kJ/m2.

Suitable drying equipment according to the present invention includes direct and indirect air dryers where the air is heated with any kind of energy source (e.g. electricity, gas, parafin, energy, etc.). Solar energy heats the air with the sun; the hot air may then be blown into an oven where the material is dried, in this case there is no UV exposure. “Drying” as used herein does not include freeze-drying.

Typically, the drying according to the invention is conducted at a temperature of from 35 to 120° C., preferably, in order to have optimum drying time, from 50 to 100° C., most preferably from 60 to 100° C.

The typical drying period according to the invention is generally at least 1 hour and may be up to two weeks, preferably from 1 to 72 hours, most preferably from 3 to 48 hours.

The cut plants are typically dried to a residual moisture content of less then 15% by weight, preferably less than 10% by weight, more preferably less than 5% by weight. The residual moisture content can be measured using standard gravimetric techniques or Karl Fischer titration.

The obtained dried plant material, preferably in the form of small pieces or flakes, has a mean total content of steroidal glycosides of at least 1.3% by weight, preferably of at least 1.6% by weight. The total steroidal glycoside content from an individual plant is, according to the invention, at least 1.6% by weight, preferably at least 1.8% by weight. The dried plant material obtainable according to the invention comprises the steroidal glycoside of Formula 2—described hereinbelow—in an amount of at least 0.08% by weight (calculated as a mean), preferably 0.1%, or an individual total level of the steroidal glycoside of Formula 2 of at least 0.08% by weight, preferably 0.1%.

According to an especially preferred embodiment, the steroidal glycoside has the general structural formula (1) or a salt or ester thereof:

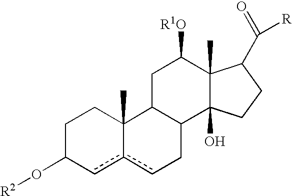

wherein

### R=alkyl;

### R¹═H, alkyl, tiglyol, benzoyl or any other organic ester group;

R²═H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

Particularly preferred steroidal glycosides are analogs of Compound of Formula 1, including Compounds of Formula (2) through Formula (8), and mixtures thereof (Me═CH₃).

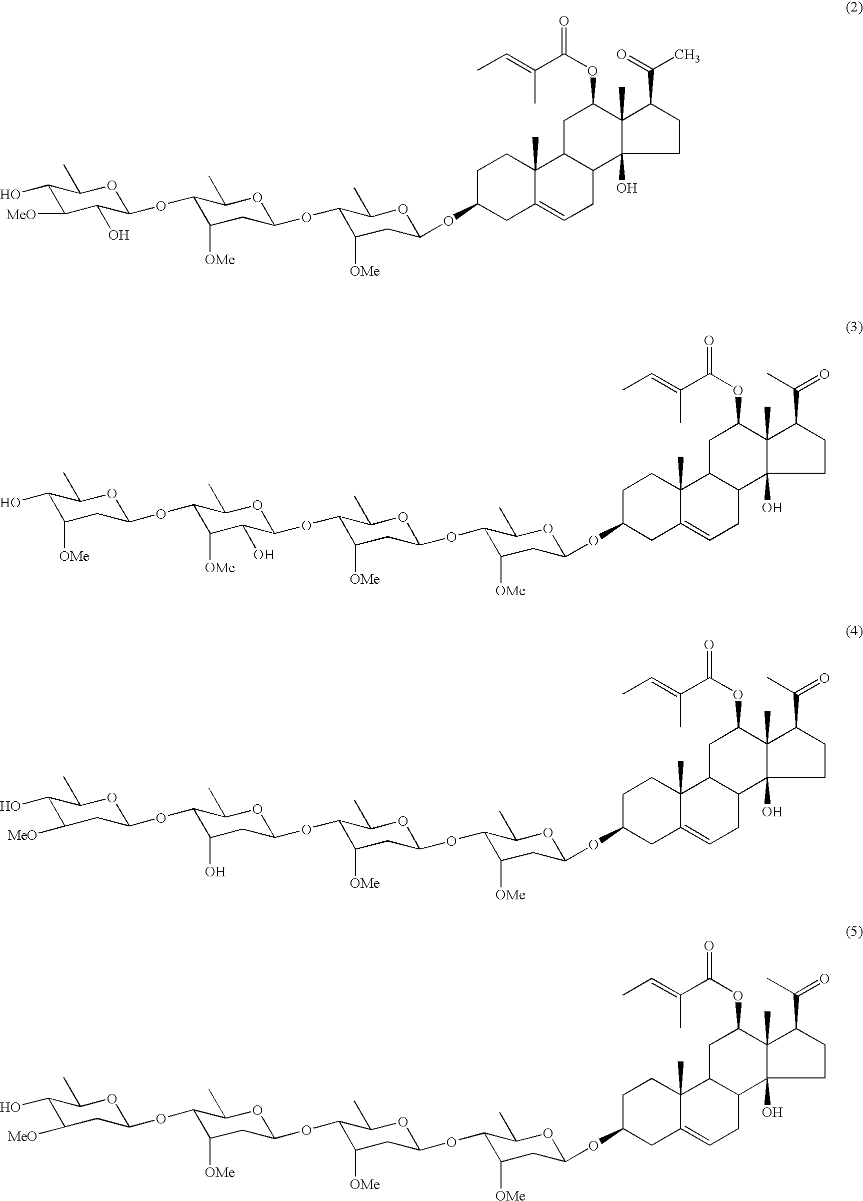

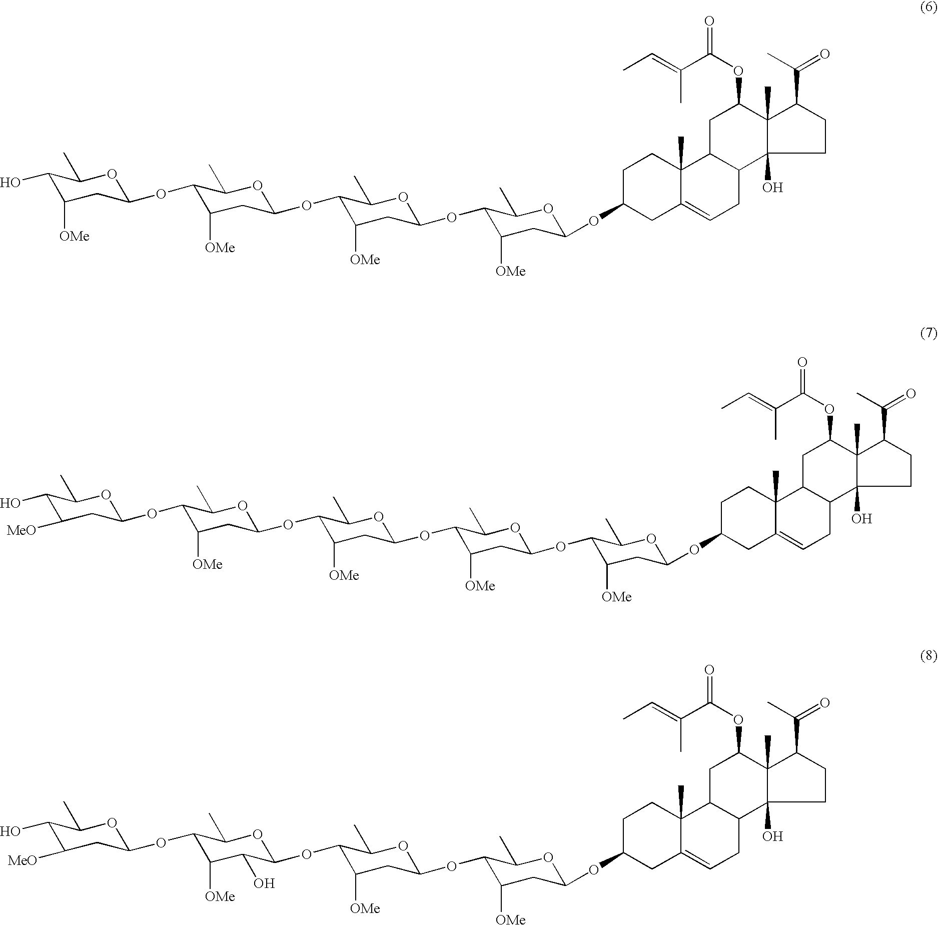

Other steroidal glycosides not specifically mentioned herein may be included in the inventive product. It will be understood that the invention also encompasses isomers, derivatives, salts, esters and analogs of the steroidal glycosides (preferably, biologically active) and mixtures thereof.

Steroidal glycoside concentrations are determined using high performance liquid chromatography (HPLC) with UV detection after extraction or dissolution.

In case of dried plant material approximately 5 g of material is refluxed with approx. 80 ml of boiling methanol for 1 hour. The resulting extract is filtered and the solid material is washed with methanol. The combined filtrate and washing are transferred to a 100 ml flask and made to volume with methanol. 1 ml of the filtrate is evaporated to dryness and reconstituted in 1 ml acetonitrile/water (50/50 v/v).

In case of extracts approximately 20 mg of the material is dissolved in 50 ml of methanol by sonication for 10 minutes. After filtration 1 ml of the filtrate is evaporated to dryness and reconstituted in 1 ml acetonitrile/water (50/50 v/v).

Steroidal glycosides are measured by LC-UV at 220 nm. To this end 20 μl of the extracts are injected onto a Zorbax RX-C8 analytical column of 250×4.6 mm packed with 5 μm particles and equipped with a Zorbax RX-C8 guard column of 12.5×4.6 mm packed with the same stationary phase. The column system is held at 40° C. Gradient elution is performed starting at 41.2% acetonitrile/methanol (85/15 v/v) and 58.8% water/methanol (85/15 v/v) at a flow rate of 1 ml/min. Initial conditions are held for 10 minutes before being linearly increased to 88.2% acetonitrile/methanol (85/15 v/v) and 11.8% water/methanol (85/15 v/v) over 30 minutes. After a final hold of 5 minutes the system is re-equilibrated to the starting conditions.

Compound of Formula 2 of any known purity (95% was used in this case) is used for calibration. Compound 2 may be isolated from an extract of dried Hoodia gordonii using preparative liquid chromatography or may be synthesized (see e.g. U.S. Pat. No. 6,376,657, incorporated by reference herein). A stock solution at 100 μg/ml is prepared in acetonitrile/water (1/1 v/v) and further dilutions are prepared to yield additional calibration standards at 75, 50, 20, 10 and 5 μg/ml. UV response at 220 nm is used for quantification against the Compound 2 calibration line. Relative response factors based on molecular weight are used to quantify the steroidal glycosides against the Compound 2 calibration line. Steroidal glycosides are defined as all peaks eluting after 15 min that are not present in the blank acetonitrile/water (1/1 v/v) sample. For the compounds of Formula 2-8 the specific relative retention times and response factors, are summarized in Table 1.

TABLE 1
Relative retention times and response factors
of some steroidal glycosides

| | Relative retention time vs. | Response factor vs. |
|---|---|---|
| Compound | Compound 2 | Compound 2 |
| formula 2 | 1.000 | 1.000 |
| formula 8 | 1.066 | 1.164 |
| formula 3 | 1.128 | 1.164 |
| formula 4 | 1.191 | 1.130 |
| formula 5 | 1.292 | 1.146 |
| formula 6 | 1.328 | 1.146 |
| formula 7 | 1.399 | 1.309 |

The other steroidal glycosides peaks eluting after 15 minutes have a response factor of 1.081 vs. Compound 2.

In a further embodiment of the process of the present invention, one or more steroidal glycosides are extracted from the dried plant material. Any extraction method may be employed. For instance extraction may be conducted as described in U.S. Pat. No. 6,376,657, incorporated by reference herein. The solvents specifically mentioned to perform the extraction are one or more of methylene chloride (dichloromethane), water, methanol, hexane, ethyl acetate or mixtures thereof. Alternatively, the steroidal glycosides may be extracted using liquid or supercritical carbon dioxide such as described in WO2005/116049 (Unilever).

The dried plant material or the extract therefrom can be used in appetite suppressant food products, and this constitutes a third aspect of the present invention. Examples of such food products are beverages, snacks, bars, spreads, dressings, soups, etc., or meal replacement products, which can be used in the management of body weight or in the dietary control of obesity.

All amounts, parts, ratios and percentages used herein are by weight, unless otherwise specified.

While the above summarizes the present invention, it will become apparent to those skilled in the art that modifications, variations and alterations may be made without deviating from the scope and spirit of the present invention as described and claimed herein. The invention will now be further illustrated in the following, non-limiting examples.

### EXAMPLE 1 AND COMPARATIVE EXAMPLE A

This experiment compared steroidal glycoside levels of oven dried (normal oven at 70° C.) (Example 1 within the scope of the invention) and sun dried (Example A outside the scope of the invention) Hoodia gordonii plants. 20 similar plants were harvested and individually chopped (bowl cutter) and dried. 10 of these were oven dried for up to 32 hours, until constant weight was obtained. The other 10 were chopped using a bowl cutter and sun dried for up to 32 hours at 40° C., until constant weight was obtained. The results that were obtained are summarized Table 2 below.

TABLE 2

| | Total Steroidal Glycosides | | Compound of Formula 2 | |
|---|---|---|---|---|
| | (% w/w, anhydrous) | | (% w/w, anhydrous) | |
| | | EXAMPLE | | EXAMPLE |
| | EXAMPLE 1 | A | EXAMPLE 1 | A |
| | Oven Dried | Sun dried | Oven Dried | Sun dried |
| Plant | 0 kJ/m² | 5.8 kJ/m² | 0 kJ/m² | 5.8 kJ/m² |
| 1 | 1.580 | 1.035 | 0.156 | 0.050 |
| 2 | 2.899 | 0.956 | 0.248 | 0.037 |
| 3 | 1.661 | 0.712 | 0.107 | 0.058 |
| 4 | 1.761 | 1.288 | 0.071 | 0.066 |
| 5 | 1.556 | 1.131 | 0.066 | 0.034 |
| 6 | 1.711 | 1.536 | 0.061 | 0.034 |
| 7 | 0.907 | 0.911 | 0.062 | 0.028 |
| 8 | 1.601 | 0.902 | 0.114 | 0.051 |
| 9 | 2.020 | 1.230 | 0.182 | 0.041 |
| 10 | 1.708 | 1.111 | 0.096 | 0.077 |
| Mean | 1.740 | 1.081 | 0.117 | 0.047 |
| Highest | 2.899 | 1.536 | 0.248 | 0.077 |
| Lowest | 0.907 | 0.712 | 0.061 | 0.028 |
| Variance | 0.245 | 0.054 | 0.004 | 0.0003 |
| Standard | 0.495 | 0.233 | 0.062 | 0.016 |
| Deviation | | | | |

It can be seen from the results in Table 2 that by limiting the exposure to UV light according to the invention, the steroidal glycoside content (both total and that of Compound 2) was substantially increased.

### EXAMPLES 2, 3 AND COMPARATIVE EXAMPLE B

This experiment compared active levels of steroidal glycosides from Hoodia plants from oven, solar oven dried (Examples 2 and 3, respectively, within the scope of the invention) and sun dried (Example B outside the scope of the invention). The solar oven is a piece of equipment covered with UV protective plastic. The air inside the oven is heated by solar radiation, without exposing the plants to UV radiation. 20 similar plants were harvested and chopped using a bowl chopper to form a homogenous batch. The batch was split over three parts which were dried in three different ways till constant weight. In the oven, the drying was conducted for 48 hours at 70° C., for solar oven the maximum outside air temperature was 37° C. and the drying was conducted for 10 days, for sun drying maximum outside air temperature was 37° C. and took 10 days. The results that were obtained are summarized in Table 3.

TABLE 3

| | Example 2 | Example 3 | Example B |
|---|---|---|---|
| | Oven, 0 kJ/m² | Solar oven, 0 kJ/m² | Sun, 5.8 kJ/m² |
| Compound of | 0.10 | 0.10 | 0.071 |
| Formula 2 | | | |
| Total steroidal | 1.6 | 1.6 | 1.2 |
| glycosides | | | |

It can be seen from the results in Table 3 that by limiting the exposure to UV light according to the invention, the steroidal glycoside content (both total and that of Compound 2) was substantially increased.

It will be apparent that for commercialization the previously mentioned process steps may be scaled up to the appropriate process and equipment sizes, types and standards practised in the particular or relevant food or agricultural industry.

It should be understood that the specific forms of the invention herein illustrated and described are intended to be representative only and that certain changes may be made therein without departing from the clear teachings of the disclosure.

1. Process for preparing a composition comprising one or more active steroidal glycosides, comprising the steps of:
(a) harvesting Hoodia plants,
(b) cutting up the harvested plants,
(c) drying the cut plants,
whereby direct exposure to UV light during the drying step is avoided, such that the total UV dose is less than about 0.5 kJ/m², to obtain dried plant material.

2. Process according to claim 1, wherein the cut plants are dried to a residual moisture content of less than about 15% by weight.

3. Process according to claim 1, wherein the total UV dose is less than about 0.35 kJ/m2.

4. Process according to claim 1, wherein the total UV dose is less than about 0.2 kJ/m2.

5. Process according to claim 1 wherein the drying is conducted with a drying equipment which is an air dryer.

6. Process according to claim 5 where the air in the air dryer is heated with an energy source selected from the group consisting of electricity, fossil fuel, ethanol, gas, parafin, solar energy, and mixtures thereof.

7. Process according to claim 1 wherein the drying is conducted at a temperature of from about 35° C. to about 120° C.

8. Process according to claim 1, wherein the plants are selected from the group consisting of Hoodia gordonii, Hoodia currorii, Hoodia Iugardii and mixtures thereof.

9. Process according to claim 8, wherein the plant is Hoodia gordonii.

10. Process according to claim 1, wherein the dried plant material comprises one or more steroidal glycosides having the formula: 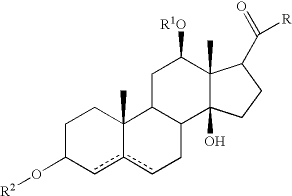
wherein R=alkyl;
R1═H, alkyl, tiglyol, benzoyl or any other organic ester group;
R2═H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6, or mixtures thereof.

11. Process according to claim 1, wherein the dried plant material comprises a steroidal glycoside selected from the group consisting of steroidal glycosides having the formula (2) through (8) and mixtures thereof:

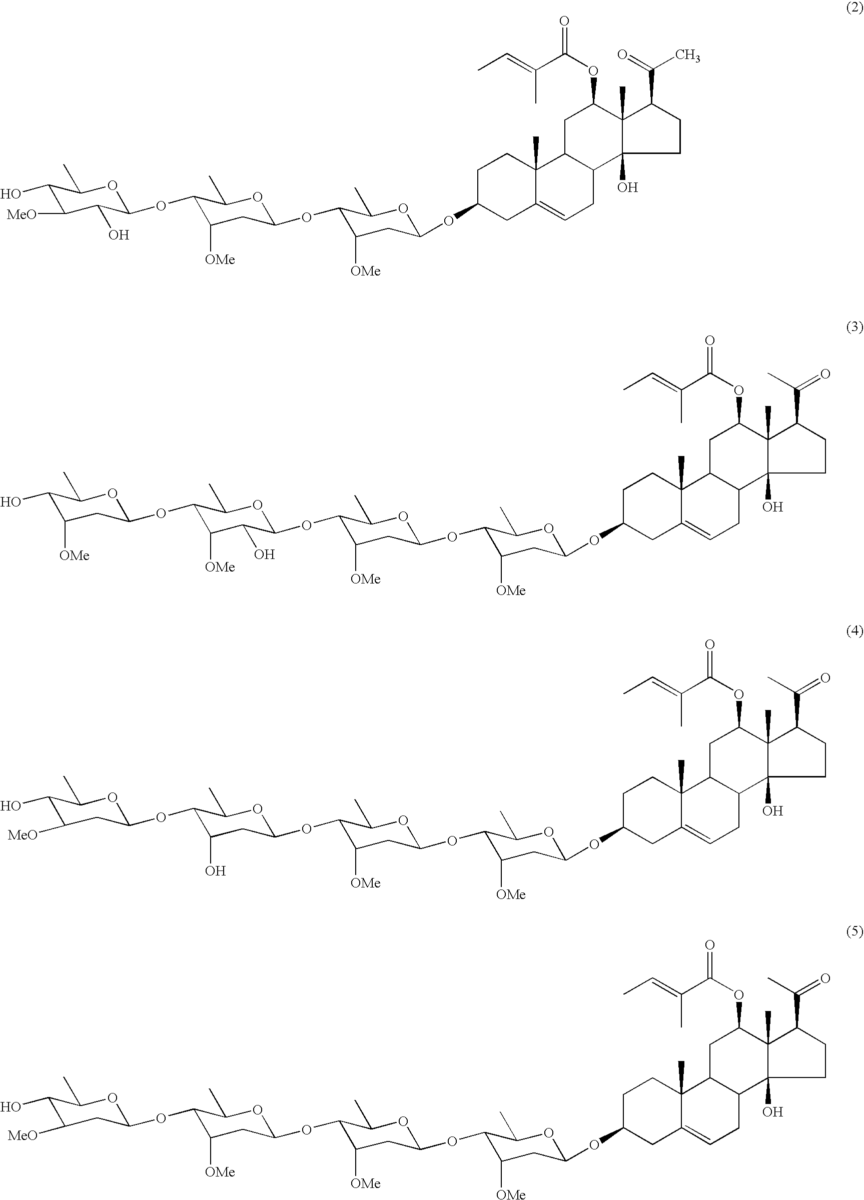

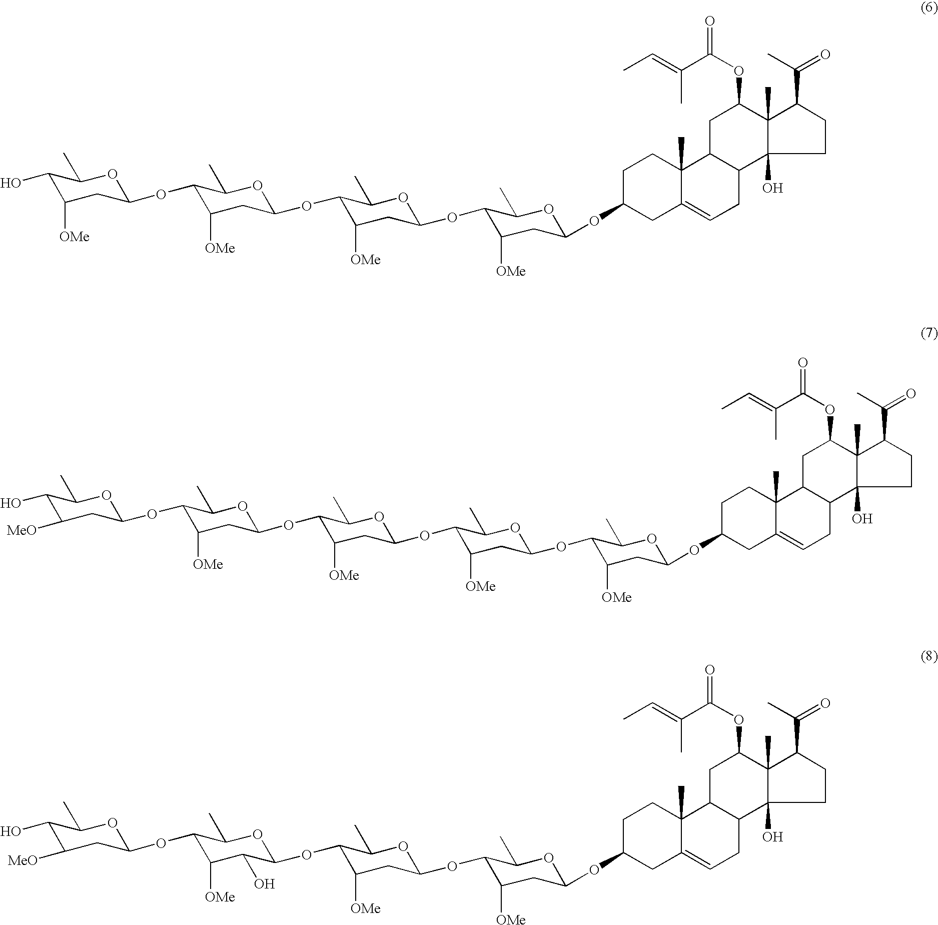

12. Process according to claim 1 wherein the dried plant material has a mean total steroidal glycoside content of at least about 1.3% by weight, based on anhydrous dried plant material.

13. Process according to claim 1 wherein the dried plant material has a mean total steroidal glycoside content of at least about 1.6% by weight, based on anhydrous dried plant material.

14. Process according to claim 1 wherein the individual dried plant material has a total steroidal glycoside content of at least about 1.6% by weight, based on anhydrous dried plant material.

15. Process according to claim 1 wherein the dried plant material comprises at least about 0.08% by weight of the steroidal glycoside of Formula 2, based on anhydrous dried plant material.

16. Dried plant material obtained according to the process of claim 1.

17. Food product comprising the dried plant material obtained according to the process of claim 1.

18. Process according to claim 1, further comprising obtaining an extract from the dried plant material.

19. An extract obtained from the dried plant material of claim 1.

20. Food product comprising an extract obtained from the dried plant material of claim 1.

21. Food product comprising an extract obtained from the dried plant material of claim 1, wherein the food product is in the form of a beverage, snack, bar, spread, dressing, soup or meal replacement product.
